(19) United States
(12) Patent Application Publication
(10) Pub. No.: US 2026/0363234 A1
(43) Pub. Date: Nov. 19, 2026

(54) METHOD OF STORING AND PRESERVING POLLEN IN A BREATHABLE VENTILATED CONTAINER

(71) Applicant: PowerPollen, Inc., Ames, IA (US)

(72) Inventors: Alex Austin, Ames, IA (US); Jason Cope, Ankeny, IA (US); Federico Valverde, Polk City, IA (US); Mark Westgate, Ames, IA (US)

(73) Assignee: PowerPollen, Inc., Ames, IA (US)

(21) Appl. No.: 19/673,082

(22) Filed: May 11, 2026

Related U.S. Application Data
(60) Provisional application No. 63/802,840, filed on May 9, 2025.

(51) Int. Cl. A01N 3/00; A01H 1/02; B65D 33/01; B65D 81/26

(52) U.S. Cl. CPC A01N 3/00; A01H 1/02; B65D 33/01; B65D 81/263

(57) ABSTRACT

Disclosed is a method of preserving the integrity and viability of collected pollen by storing the pollen in a container that provides substantial ventilation to the collected pollen during storage. The invention also encompasses introducing the collected pollen to one or more diluents to create a pollen-diluent mixture, storing the pollen or pollen-diluent mixture in a storage container, storing said storage container in a controlled environment, venting the storage container during the storage period, and applying the pollen to one or more female plants to pollinate the female plants after the end of the storage period.

This invention relates generally to a novel method for storing and preserving pollen in a breathable container that increases the overall viability and fertility of pollen for use in pollinating plants. The invention may be conducted with either fresh pollen, partially dried pollen, or pollen that has previously been stored or preserved.

### BACKGROUND

The current invention has application to the field of pollen longevity and viability. Pollen can be classified as either orthodox or recalcitrant, based on the water content at the time of dispersal, sensitivity to desiccation, accumulation of biochemical components, and morphology of the pollen grain (Pacini & Dolferus (2016) in Understanding Reproductive Stage Stress Tolerance, Abiotic and Biotic Stress in Plants-Recent Advances and Future Perspectives, ed. A. Shanker (London: Intech), 703-754). Mature pollen grains in orthodox species are typically desiccated at the time of their dispersal and generally have less than 30% water by weight, range in size from 30 to 100 μm, and have one to six furrows and pores on the pollen grain. Examples of orthodox pollen species include Fabaceae species (beans) and most Lamiaceae species (mint, rosemary and other herbs) and many trees, such as maples and oaks. In recalcitrant pollen species, the pollen typically is released with large percentage of water (greater than 30% by weight) and the pollen desiccates on its journey to the female flowers. Recalcitrant pollen is typically 15 to 30 μm or 70 to 150 μm in diameter, has no furrows, and 0-12 (or more) pores on the grain. Examples of recalcitrant pollen species include almost all Poaceae species, squash and pumpkins (Cucurbita pepo), and spinach (Spinacia oleracea) as well as some trees, such as birch (Franchi et al. (2011) J. Exp. Bot. 62:15 5267-5281).

Particularly in regard to recalcitrant pollen, longevity is significantly influenced by environmental conditions, most notably temperature and relative humidity. Pollen, which is naturally shed from the flowers or flowering structures of angiosperms, is subject to rapid loss of viability at various rates depending on the environmental conditions and type of pollen (orthodox/recalcitrant), once it is shed from the plant. While typically more resilient than recalcitrant pollen in most environmental conditions, orthodox pollen can also suffer loss of viability due to lack of respiration, which prevents the dispersal of gas buildup that can have deleterious effects on viability. For recalcitrant pollen, viability can be lost in minutes to hours depending on species and environmental conditions. Exposure to dry air and high temperature is particularly detrimental to pollen viability and longevity once it is shed from the plant. Thus, under natural field conditions, pollen has a limited lifespan during which it remains viable, referred to in this application as the “viability window”, as provided herein below. In particular, pollen from the Poaeceae (Gramineae) family of plants, commonly referred to as grasses, is particularly vulnerable and short-lived (Barnabas & Kovacs (1997) In: Pollen Biotechnology For Crop Production And Improvement. (1997). Sawhney, V. K., and K. R. Shivanna (eds). Cambridge University Press. pp. 293-314). This family of plants includes many economically important cereal crops, including maize.

Methods to improve pollen viability and extend the duration of its viability are of significant value to the agricultural industry. Specifically, if pollen collected from plants can be stored in a viable state for a period of time, this pollen may be used to pollinate female flowers as desired in a number of advantageous ways. Using stored pollen allows for pollination which is not dependent on active pollen shed, temporal synchrony with pistil (female flower) receptivity, use of male sterility, and/or physical isolation from other pollen sources. Currently, many species rely on self-pollination or cross pollination by neighboring plants to produce seed. Typically, in the agricultural hybrid seed industry, mechanical, physical, and/or genetic interventions are required to ensure female plants are cross pollinated by pollen of a specific genetic constitution to produce hybrid seed. Such measures, for example, are used routinely to produce hybrid maize and rice seed. In some crops, however, even these measures do not ensure cross pollination by a specific desired pollen source. Currently, it is challenging to produce such crops commercially as hybrids, although research continues in this regard. Examples of these crops include, but are not limited to, wheat and soybean.

Many attempts have been made to preserve pollen and extend its viability for pollinations beyond the time the pollen would remain viable if left exposed to uncontrolled ambient conditions. Among the grasses, studies with maize are exemplary of the progress made in pollen preservation. Many types of treatments have been tested for maintaining or extending maize pollen viability and/or fertility. Among them, the benefit of storing maize pollen at high humidity and/or cold temperature has been reported by many.

Among the earliest accounts of maize pollen preservation, it was reported that pollen died in two to four hours in the absence of controlled environmental storage conditions (Andronescu, Demetrius I., The physiology of the pollen of Zea mays with special regard to vitality. Thesis for degree of Ph.D. University of Illinois. 1915).

Various methods have been used since then in an attempt to improve maize pollen preservation efficacy. These include raising the relative humidity of the storage environment and storage at low temperatures (Pfahler, P. L. and Linskens, H. F., (1973) Planta, 111(3), pp. 253-259; Frova, C. B. and Feder, W. A., (1979) Ann Bot, 43(1), pp. 75-79). When high humidity (90% RH) and low temperature (4 °C) during storage are combined, the capacity of maize pollen to germinate on artificial media is partially maintained for eight days (Sartoris, G. B., (1942) Am J Bot, pp. 395-400). In some cases, it may be desirable to dehydrate pollen to various degrees, such as by vacuum drying or exposing pollen to a vapor pressure deficit that causes water to diffuse out of the pollen (Jackson, M. A. and Payne, A. R. (2007) Biocontrol Sci Techn, 17(7), pp. 709-719), Greenspan, L., (1977) J Res Nat Bur Stand, 81(1), pp. 89-96).

Desiccation is known to have a direct impact on pollen viability. Barnabas ((1985) Ann Bot 55:201-204) and Fonseca and Westgate ((2005) Field Crops Research 94:114-125) demonstrated that freshly harvested maize pollen could survive a reduction in original water content of approximately 50%; few pollen grains retained viability or capacity for normal pollen tube formation with further water loss beyond that level. Other methods of storing pollen for varying periods of time are known in the art, including freeze-drying, vacuum-drying, and storage in organic non-polar solvents. Limitations in the scalability of these pollen preservation techniques combined with the complex, non-portable equipment requirements, however, render these techniques completely impractical for large-scale use because they are unable to accommodate the large volumes of pollen necessary for field-scale applications.

U.S. Pat. No. 5,596,838 from Greaves, et al., discloses a method of storing pollen that involves a reduction in moisture level by exposing pollen to reduced atmospheric pressures prior to storage. This technique prepared small quantities of pollen, such as from a single maize plant, for subsequent storage under sub-zero conditions. The methodology and mechanical system requirements, however, lacked the capacity to produce stored pollen in quantities large enough to enable commercial seed or grain production applications. These requirements effectively negated any opportunity to advance the technology beyond research level investigations. Another Greaves et al. patent, U.S. Pat. No. 6,141,904 provides that pollen may be placed in a carrier material to increase flowability, such as a flowable powder. In addition, U.S. Pat. No. 2,669,066 suggests mixing pollen with substances having a high protein content for treatment and application, such as powdered egg albumin, powdered casein, or powdered milk in instances of short-term storage. Other prior art, such as U.S. Patent Application Publication No. 2025/0287,938 to Carter et al., provides for the storage of pollen in limited quantities, but fails to provide pollen capable of high pollination efficiency, dramatically reducing the utility of such methods.

The availability of preserved viable pollen overcomes many of the production challenges faced by the hybrid seed industry. With respect to hybrid seed production, the availability of stored pollen for delivery to female flowers can eliminate many standard, costly practices of commercial seed production including. These include, but are not limited to planting male plants in proximity to, but separate from, female plants to enable hybridization; increasing field area allocated to male parent plants due to their low pollen production; isolating female parent plants from undesired pollen sources; and use of genetic, chemical, or mechanical male sterility of the female plants. These standard, but costly practices divert field space and resources away from female parent plants which produce the seed. Eliminating any one of these practices would have an immediate positive impact on seed yield per acre. Moreover, stored pollen can be applied at any time female plants are receptive to pollen.

Timely coincidence of pollen shed from male parent plants and receptivity of female parent plants is of great importance to economically viable hybrid seed production. Occasionally, the reproductive development of male and female parent plants fails to coincide as planned due to management, environment, or natural genetic variation. In such cases, application of preserved, viable pollen ensures pollination of female parent plants at the optimal time. Pollination by undesired external (adventitious) sources of pollen or undesired self-pollination of female parent plants also can be reduced or eliminated by applying stored pollen of the desired genetics at the most appropriate time. Furthermore, production-level fields for parent seed increase are typically an acre or more in size, while hybrid production fields can be hundreds of acres in size. Hybrid seed production fields typically have between 30,000 to 40,000 female plants per acre. Such fields require kilograms of pollen for efficient pollination, thereby demonstrating the need for methods that enable preservation and storage of large quantities of pollen.

Moreover, mixing pollen with a diluent can extend the viability of pollen storage by limiting deleterious pollen-pollen interactions. Diluents also can improve the uniformity of pollen delivery to receptive female flowers thereby increasing seed set, regardless of whether pollen is applied by hand or by machine. In addition, using diluents enables pollen to be applied in less-than-saturating doses to increase pollination efficiency and to distinguish effects of experimental treatments. Maintenance of pollen in pollen-diluent mixtures as large as 12,385 cm³ (1,537 g pollen) in one storage container, for example, would enable mechanical pollination of nearly two acres of hybrid seed production. Numerous diluents have been described in the art including those disclosed in U.S. Pat. No. 12,245,587.

Today, the genetic and commercial potential of a particular hybrid seed product is determined at the beginning of a growing season by the genetics of the pollen-donating male parent and pollen-receiving female parent once they are planted together. Using the embodiments of the present disclosure, however, a hybrid seed producer responding to changing market opportunities can decide at the time of pollination to use a different male parent pollen (i.e., genetic source) for pollination to produce more valuable hybrid seed. Thus, stored pollen can be used to deliver unique genetic traits or genes that enhance various aspects of the seed. For example, traits might be delivered for resistance to select insect pests that currently are prevalent in the area. Importantly, the embodiments of the present disclosure also can ensure a high level of genetic purity in the hybrid seed. Furthermore, the pollen preserved using the embodiments of the invention can be used to supplement self- or sib-pollen in grain production, or to deliver traits in cross-pollinated grain production. Embodiments of the invention may also be used for research purposes, or for the production of fruit or vegetables.

### BRIEF DESCRIPTION OF THE FIGURES

FIG. 1 shows a mesh bag used in embodiments of the present invention containing pollen-diluent blend set in a storage bin.

FIG. 2 shows experimental results demonstrating the effect of active ventilation on the viability of stored pollen.

FIG. 3 shows experimental results demonstrating the effect of passive ventilation on the viability of stored pollen.

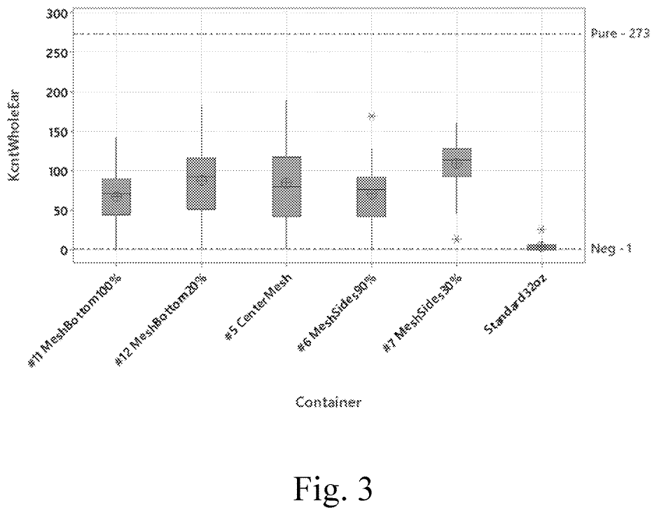

FIG. 4 shows experimental results demonstrating the effect of passive ventilation on the viability of stored pollen.

FIG. 5 shows experimental results demonstrating the effect of bulk ventilated storage on the viability of stored pollen after two days.

FIG. 6 shows experimental results the effect of bulk ventilated storage on the viability of stored pollen after three days.

FIG. 7 shows experimental results demonstrating the effect of bulk ventilated storage on the viability of stored pollen after four days.

FIG. 8 shows a storage container with a ventilated column used in some embodiments of the present invention containing a pollen-diluent blend.

FIG. 9 shows a storage container with a ventilated column used in some embodiments of the present invention containing a pollen-diluent blend.

FIG. 10 shows a mesh bag used in some embodiments of the present invention containing a pollen-diluent blend.

FIG. 11 shows experimental results demonstrating the effect of ventilated storage container configurations on the viability of stored pollen over three days in storage.

FIG. 12 shows experimental results demonstrating kernels per ear resulting from one application of a limiting amount of pollen-diluent mixture stored for two days in ventilated containers of various volumes and configurations.

FIG. 13 shows kernels per ear resulting from one application of a limiting amount of pollen-diluent mixture stored for four days in ventilated containers of various volumes and configurations.

FIG. 14 is a flow chart view of an embodiment of the method of the present invention.

### SUMMARY OF THE INVENTION

Provided is a method of preserving pollen comprising mixing collected pollen with a diluent, storing the pollen-diluent mixture in storage containers of various configurations including, but not limited to, primarily solid surface containers having a breathable barrier over some portion of the surface area of the container or primarily non-solid surface containers having a permeable barrier over all or the majority of the surface area of the container, such as mesh bags, and venting at least some portion of the surface area of the storage containers during storage, wherein said preserved pollen maintains its viability and can be used to successfully pollinate plants. The method includes storing the pollen mixed with diluent. The storage can take place at a temperature ranging from −196 degrees Celsius to −80 degrees Celsius, at a temperature ranging from −80 degrees Celsius to 0 degrees Celsius, or at a temperature ranging from 0 degrees Celsius to 25 degrees Celsius.

### GLOSSARY

“Female parent plant” or “female plant” is used to mean a plant that is being used as the recipient of pollen, and which has receptive flowers that are passively or intentionally being pollinated.

“Fertile” or “fertility” is used to describe the ability of pollen to deliver the sperm nuclei to the ovule and thereby effect double fertilization. In flowering plants, each pollen grain contains two sperm nuclei. The term “double fertilization” refers to one sperm nucleus fusing with the polar nuclei to produce the endosperm tissues, and the other sperm nucleus fusing with the egg nucleus to produce the embryo.

“Fertilization event” within the ovule means the successful joining of one sperm nucleus with the egg cell nucleus to initiate embryo development and one sperm nucleus joining with the two polar nuclei outside the egg cell to initiate endosperm development.

“Fresh” when applied to pollen means pollen released from the anthers of a flower which, in its natural pattern of organ growth and development, releases pollen upon anther dehiscence in response to maturity and promotive environmental conditions. Due to the environmental sensitivity described, recalcitrant pollen will generally cease to be fresh approximately fifteen minutes after release, corresponding with the close of the viability window.

“Germinability” refers to the ability of pollen to germinate and form a pollen tube.

“Longevity,” when applied to pollen, is used describe the length of time that pollen remains both viable and fertile.

“Loss of viability” is a pollen characteristic that means the viability level of the pollen has fallen to a level below that required for successful initiation of germination.

“Loss of fertility” is a pollen characteristic that means that the fertility level of the pollen has fallen to a level below that required for successful germination and delivery of two sperm nuclei to the ovule and thereby effecting double fertilization.

“Male parent plant” or “male plant” means a plant from which pollen is collected from for use in pollinations.

“Preservation” means any treatment or storage of collected pollen that results in a level of viability, fertility, or both, which is greater than the level of viability, fertility, or both, which would occur if the pollen were held in unregulated conditions.

“Pollination efficiency” is a measure of the success of a pollination event relative to the amount of pollen applied whether pollination occurs naturally or intentionally. In the context of pollen delivery to female plants, pollination efficiency is measured as the number of pollen grains captured by the stigmatic tissue calculated on a per flower, floral rachis, or per plant basis resulting from the pollination event. In the context of seed formation, pollination efficiency is measure as the number of pollen grains applied per seed produced calculated on an ovule, ovary, rachis, or plant basis resulting from the pollination event. In the context of maize, pollination efficiency is a measure of the number of pollen grains applied per ear divided by the number of kernels per ear resulting from the application.

“Storage” means any period of pollen containment with the intent of using the pollen at a later time and/or date.

“Successful fertilization event” means the fusion of male and female gametes producing a viable zygote capable of developing into an embryo. The observable hallmarks of a successful fertilization event are formation of a viable zygote, development of endosperm (in angiosperms), ovule maturation into a seed, fruit set, and ultimately a seed capable of germinating into a new sporophyte generation.

“Vented surface area” is used to describe the surface area of a given storage container that enables gas exchange between a pollen or pollen-diluent blend within the storage container and the environment outside the storage container.

“Venting” is used to describe the process of exposing a given volume of pollen or pollen-diluent blend through a vented surface area of a storage container to enable gas exchange between the volume of pollen or pollen-diluent blend within the storage container and the storage environment in which the storage container is contained. Venting may include both passive venting that occurs from exposure of the volume of pollen or pollen-diluent blend through a vented surface area to the storage environment as well as active venting wherein one or more gasses are mechanically passed through, evacuated, or removed from the volume of pollen or pollen-diluent blend.

“Viable” or “viability” is used to describe pollen that is able to germinate and grow a pollen tube to at least a length twice the diameter of the pollen grain or pollen which has been judged viable by demonstration that the cellular nature of the material remains integral and is judged to maintain intactness such that normal cellular processes of metabolism and intracellular functioning is possible. The viability of pollen can be assessed in numerous ways, including, but not limited to, assessment of pollen tube growth on artificial media or excised stigmas or styles, assessment of cellular intactness by vital staining of numerous sorts, absence of electrolyte (e.g., potassium) leakage, and impedance flow cytometry. Viability can refer to a single pollen grain or a population of pollen grains. When a percentage value is used to describe pollen viability, the value is typically being applied to a population of pollen.

“Viability window” refers to the lifespan during which pollen remains viable when exposed to unregulated conditions.

“Vigor” refers to pollen performance, such as speed of germination and tube growth.

### DETAILED DESCRIPTION

The following is a detailed description of embodiments of technology and methods of storing and/or preserving pollen in a breathable container which enable improved and/or extended viability of collected pollen by subjecting it to specialized storage conditions. The invention encompasses the use of freshly collected pollen or previously collected and preserved pollen. The pollen may have been obtained from a single genetic source or from multiple genetic sources, which may be combined prior to preservation.

Embodiments of the invention include applying the pollen and/or pollen-diluent mixture to receptive plants. The application of the pollen and/or pollen-diluent mixture can be used to generate seeds for seed production purposes or for grain production purposes. The application of the pollen and diluent mixture can be used for other purposes, such as product creation and product research, or fruit and vegetable production that is dependent on sexual reproduction.

The diluent used in some embodiments of the invention may comprise inorganic particles, organic particles, dormancy regulators, osmotic regulators, cellular respiration inhibitors and disruptors, electron transport inhibitors, uncouplers of membrane electron transport, membrane stabilizers and conditioners, cell wall stabilizers and conditioners, growth regulators, plant hormones, cryoprotectants, monosaccharides, disaccharides, polysaccharides, anti-flocculants, dispersal agents, bentonite, starch, including but not limited to corn starch or potato starch as seen in U.S. patent application Ser. No. 19/290,668, cellulose, sand, desiccants, flow agents, mineral oil, paraffin oil, sugars in liquid, dry sugars, water, and activated charcoal. These diluents may be obtained from any source, including natural sources, chemical and transgenic sources, and synthetically derived sources.

Preserving recalcitrant (high moisture, desiccation sensitive) pollen in a dry powder diluent has proven beneficial for improving seed set when the pollen-diluent mixture is applied mechanically in seed production systems as seen in U.S. Pat. No. 12,245,587. Follow on studies have revealed, however, that a pollen-diluent mixture stored within sealed containers might limit the exchange of critical gases (e.g. O₂, CO₂, H₂O, volatile organics, etc.) between the biologically active pollen grains and the external environment, thereby curtailing the duration of pollen viability in storage.

Data from numerous laboratory and field studies now confirm that increasing the potential for exchange of gases between the pollen-diluent mixture and the environment exterior to the storage container consistently improves pollen longevity in storage as measured by seed set under field conditions. Moreover, bulk storage of recalcitrant pollen has been stymied due to the difficulty of maintaining uniform ventilation throughout large volumes of pollen and/or pollen-diluent mixtures required for commercial purposes.

According to one embodiment of the present invention, the integrity and viability of fresh pollen is preserved by collecting fresh pollen from a field, optionally introducing the fresh pollen to one or more diluents to create a pollen-diluent mixture, storing said pollen or pollen-diluent mixture in a storage container, storing said storage container in a controlled storage environment, while venting through all or a portion of the surface area of the storage container during the storage period. Pollinating receptive female plants with pollen stored in such ventilated containers resulted in improved seed set relative to that obtained using pollen stored in comparable container that did not provide adequate ventilation.

In some embodiments of the invention, the controlled storage environment is controlled for one or more different factors, including pressure, moisture, and/or temperature. In embodiments of the invention where the controlled environment is moisture controlled, the moisture control results in a controlled environment with a relative humidity between 50-100%, such as between 55-100%, 55-95%, 60-95%, 65-95%, 70-95%, 75-95%, 60-85%, 65-85%, 65-75%, 70-90%, and/or 75-85%.

In some embodiments of the invention, a plurality of pollen grains is mixed with a diluent prior to being placed in the storage container. In the preferred embodiment of the invention, the surface of the storage container is made up of a breathable material, such as a mesh bag with 55-micron sized openings suitable for retaining the pollen and/or pollen-diluent mixture while allowing for gas exchange. The breathable material surface area may comprise all or some portion of the overall surface area of the storage container. In some embodiments, the breathable material surface area comprises between 5-10% of the overall surface area of the storage container. In other embodiments, the breathable material surface area comprises other portions of the overall surface area, such as between 10-15%, 15-20%, 20-25%, 25-30%, 35-40%, 45-50%, 50-55%, 55-60%, 60-65%, 65-70%, 70-75%, 75-80%, 80-85%, 85-90%, 90-95%, such as 95-100% of the overall surface area.

In some embodiments of the invention, the breathable material surface area is configured to maintain a ratio of vented surface area to pollen-diluent volume. In one embodiment, the vented surface area to pollen or pollen-diluent volume is at least 0.03 cm⁻¹, such as at least 0.04 cm⁻¹, 0.05 cm⁻¹, 0.07 cm⁻¹, 0.08 cm⁻¹, 0.1 cm⁻¹, 0.11 cm⁻¹, 0.13 cm⁻¹, 0.14 cm⁻¹, 0.15 cm⁻¹, 0.17 cm⁻¹, 0.26 cm⁻¹, 0.44 cm⁻¹.

Embodiments of the invention optionally include a drying step prior to storage of the pollen or pollen-diluent mixture. This drying step reduces the pollen moisture content (PMC) of the pollen. In one embodiment, the PMC can be reduced to between 15-60%, such as between 15-20%, 20-25%, 25-30%, 35-40%, 40-45%, 45-50%, 50-55%, or 55-60% and all measurable PMCs in between.

Embodiments of the invention result in pollen that remains at high levels of viability for extended periods of time. This enables pollinations to occur with a high efficiency, as less pollen is required to be applied to plants to achieve the desired level of seed set or fruit formation. In one embodiment, the invention enables pollination efficiency of less than 500 (measured as pollen grains applied per ear divided by the number of kernels per ear) after a period of at least two days of storage, such as a pollination efficiency of less than 450, such as a pollination efficiency of less than 400, such as a pollination efficiency of less than 350, such as a pollination efficiency of less than 300, such as a pollination efficiency of less than 250, such as a pollination efficiency of less than 200, such as a pollination efficiency of less than 150. In other embodiments of the invention, the invention enables pollination efficiency of less than 500 after a period of at least four days, such as a pollination efficiency of less than 450, such as a pollination efficiency of less than 400, such as a pollination efficiency of less than 350, such as a pollination efficiency of less than 300, such as a pollination efficiency of less than 250, such as a pollination efficiency of less than 200, such as a pollination efficiency of less than 150, such as a pollination efficiency of less than 100, such as a pollination efficiency of less than 50, such as a pollination efficiency of less than 40, such as a pollination efficiency of less than 30, such as a pollination efficiency of less than 20, such as a pollination efficiency of less than 10, such as a pollination efficiency of less than 5.

Likewise, embodiments of the invention can include pollinations that occur with a high efficiency as measured by pollen grains required to be applied to each stigma on each flowering structure per successful fertilization event. In one embodiment, the invention enables successful fertilization events following the application of fewer than 500 grains of pollen after the pollen has been stored for a period of at least two days. In other embodiments, successful fertilization events may require fewer than 450 grains of pollen, fewer than 400 grains of pollen, fewer than 350 grains of pollen, fewer than 300 grains of pollen, fewer than 250 grains of pollen, fewer than 200 grains of pollen, fewer than 150 grains of pollen, fewer than 100 grains of pollen, fewer than 75 grains of pollen, fewer than 50 grains of pollen, fewer than 25 grains of pollen, or fewer than 10 grains of pollen to be applied to the stigma of each flowering structure to result in each successful fertilization event. In other embodiments of the invention, the pollen may have been stored for at least two days, at least three days, at least four days, at least five days, at least six days, at least 7 days, at least 2 weeks, at least 3 weeks, at least 4 weeks, at least 2 months, at least 3 months, at least 4 months, at least 5 months, at least 6 months, at least a year, or longer, especially in the case of orthodox pollen species.

While the storage container with an exterior surface area made up of a breathable material has been described as a mesh bag with 55-micron sized openings in some embodiments, in other embodiments the storage container may be a mesh bag with different size openings to accommodate variation in pollen size across plant species and diluent formulations, with openings of the mesh bag smaller in size than the average diameter of the pollen grains or diluent mixture to be stored in the storage container. In some embodiments, the storage container may be a mesh bag constructed to be a specific geometrical shape such as, but not limited to, a cylindrical or cuboidal. The portion of the exterior surface area of the storage container that is made up of a breathable material may be constructed of any type of barrier capable of physically separating the pollen or pollen-diluent blend from the environment while enabling gas exchange with the storage environment. Such barrier can be made of synthetic mesh, metal screens, natural or synthetic fabrics, open cell foam, and other “breathable” materials.

The mesh bag of some embodiments of the present invention may also include ribbing located on the exterior surface to help maintain its geometric shape. The mesh bags of some embodiments of the present invention may also include a bottom opening for ease of pollen removal. Such an opening may be closed by a fastening apparatus, such as a zipper, a flap that closes using a hook-and-loop material, snaps, buttons, or similar fasteners, or other fastening apparatus suitable to enable the bottom opening to be opened and closed as desired. The mesh bag may also include handles to facilitate transport when the mesh bag is filled with pollen or a pollen-diluent blend. In some embodiments, once the mesh bag is filled with pollen or a pollen-diluent mixture, the mesh bag will be stored in a manner that promotes ventilation of the pollen or pollen-diluent blend within the mesh bag, such as by placing the mesh bag on a wire rack frame, or by suspending the mesh bag from a hanging structure, such as a rod or a hook mounted at or near the ceiling of a controlled storage environment.

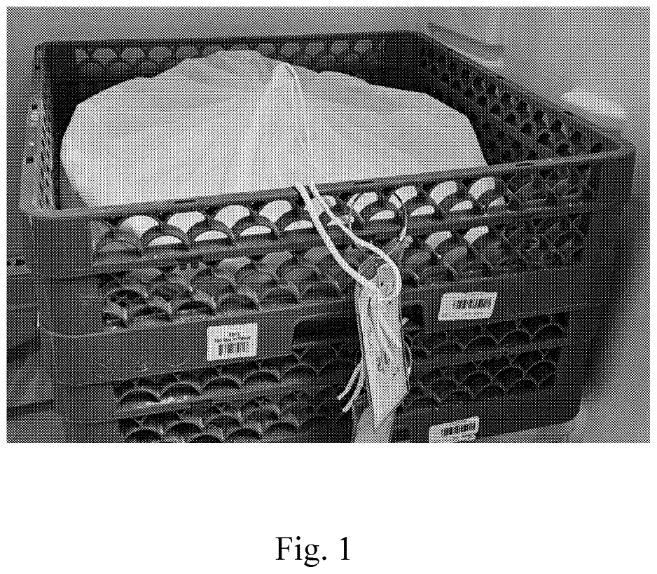

In some embodiments, the storage environment is controlled to maintain one or more variables, such as pressure within the storage environment, temperature within the storage environment, or humidity within the storage environment. In other embodiments, the mesh bags may be stored by placing them in storage bins. These storage bins are configured with a number of large openings to allow ventilation of the mesh bag stored within. Preferably, these large openings are substantially larger than the openings in the mesh bags. While the openings in the mesh bags may be 55 microns, the large openings in the storage bins may be at least a half centimeter and may range from between half centimeter to 20 centimeters. The storage bins may comprise a number of large openings of different sizes and shapes, such as circles, triangles, or rectangles. The storage bins may also be further configured to stack one atop another in an interlocking manner. The interaction between the stacking storage bins and the larger openings of the storage bins enables the mesh bags to be stacked on top of each other inside of the storage bins while maintaining adequate ventilation for each mesh bag. FIG. 1 shows a mesh bag containing a pollen-diluent mixture stored in a storage bin in accordance with one embodiment of the invention. In some embodiments of the invention, the mesh bags may be suspended in the storage bins by a rod such that the sides of the mesh bag do not touch the storage bins.

Experimental results for examples are shown in box plots that present the mean (symbol), median (—), upper and lower quartiles, and the range of the data. Where applicable, “Neg” denotes a treatment where no pollen was applied. Similarly, “Pure” denotes a treatment that comprises a 4X dose of pure fresh pollen applied.

Experimental results obtained using a “scoop and tube” method involved the targeted application of a limited dose of pollen-diluent blend directly to the silks.

### Example 1

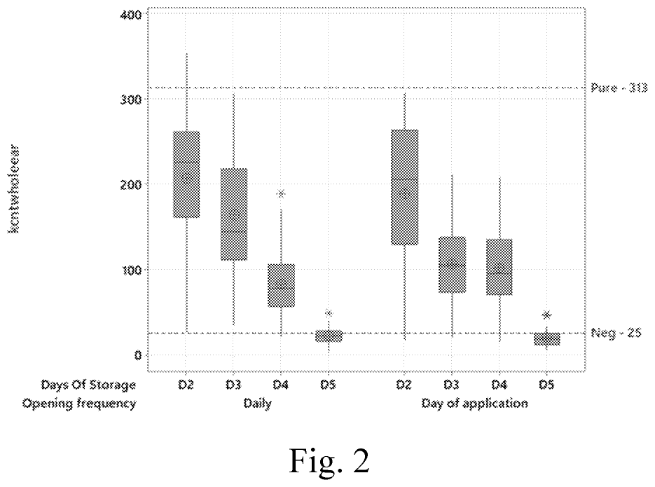

An experiment was conducted to determine whether occasional manual aeration of the head space above the pollen-diluent mixture within a sealed storage container would improve pollen longevity as assessed by pollen limited seed set on field-grown plants. Pollen was collected from maize inbred WSS644, mixed with a diluent in a 4:1 ratio of diluent:fresh pollen (w/w), and maintained at 4 °C for 2, 3, 4 or 5 days prior to being applied ( 1/64th tsp applied by scoop and tube method) to receptive silks of maize inbred W8039 grown in the field. The 0.9 L storage containers were filled with 0.54 L of pollen-diluent mix leaving 0.36 L of internal volume or ‘head space’ above the pollen-diluent mix for gas exchange. One set of storage containers remained sealed in storage until the pollen was sampled for application to the plants. A parallel set of storage containers were manually opened briefly each day to allow the gasses in the ‘head space’ to equilibrate with the atmosphere, then resealed until the day the pollen-diluent mixture was sampled for application. In both cases, the opportunity for gas exchange between the pollen-diluent mixture and the atmosphere was limited to the surface area of the pollen-diluent mix (approximately 103 cm²) and the ‘head space’ above the mixture while the storage vessel was sealed. The surface area/volume ratio for gas exchange in these containers was approximately 0.19 cm⁻¹ (103 cm²/540 cm³). FIG. 2 shows the results from this experiment. There was a rapid decrease in seed set from Day 2 (“D2”) to Day 3 (“D3”) when pollen was stored in the continuously sealed containers. Pollen lost viability completely by Day 5 (“D5”) in storage, as evidenced by seed produced per plant equivalent to unpollinated controls (Neg). Kernel set also decreased when the storage containers were opened each day in storage (“Daily” opening frequency). There was, however, a clear improvement, in kernel set over storage in sealed containers by D3. This result suggested that aeration during storage could be beneficial for promoting pollen longevity.

### Example 2

This experiment was conducted to determine whether passive ventilation of the pollen-diluent mixture within a storage container would provide a benefit for pollen longevity in storage as assessed by pollen-limited kernel set on field-grown plants. Pollen was collected from maize inbred WSS644, mixed with a diluent in a 4:1 ratio of diluent:fresh pollen (w/w), and maintained at 4 °C for 6 days in 32 oz (0.95 L) HDPE plastic containers prior to manual pollination ( 1/64th tsp applied by scoop and tube method) of receptive silks of maize inbred W8039. One set of containers (32 ozSOP) was unmodified and remained sealed without ventilation throughout storage. Other storage containers were modified to various degrees by replacing portions of the container walls with 50-micron nylon mesh to enable gas exchange between the pollen-diluent mix and the external environment. Ventilated modifications included: 100% Mesh Bottom Mesh, 20% Bottom Mesh, Center Column Mesh Column, 90% Mesh Sides, and 30% Mesh Sides.

TABLE 1
Parameters of non-ventilated and partially ventilated
storage containers used to assess impact of passive
ventilation on viability of pollen in storage.

| | | | | | Vented |
|---|---|---|---|---|---|
| | | | | | Surface |
| | | | | Container | Area: |
| Storage | Surface | Surface | | SA: Pollen- | Pollen- |
| Container | Area | Area | Pollen- | Diluent | Diluent |
| (0.95 L, | Ventilated | Ventilated | Diluent | Volume | Volume |
| 525 cm²) | (cm²) | (% total) | (cm³) | (cm⁻¹) | (cm⁻¹) |
| 90% mesh | 287 | 55 | 645 | 0.81 | 0.44 |
| sides | | | | | |
| 100% mesh | 103 | 20 | 605 | 0.87 | 0.17 |
| bottom | | | | | |
| 30% mesh | 96 | 18 | 685 | 0.77 | 0.14 |
| sides | | | | | |
| Center | 71 | 14 | 645 | 0.81 | 0.11 |
| Column | | | | | |
| Mesh | | | | | |
| 20% mesh | 21 | 4 | 725 | 0.72 | 0.03 |
| bottom | | | | | |
| No container | 0 | 0 | 405 | 1.29 | 0.0 |
| ventilation | | | | | |
| (Standard | | | | | |
| 32 oz) | | | | | |

After 6 days of storage at 4 °C, pollen stored in the non-ventilated containers (Standard 32 oz in the figure), failed to produce any kernels. Pollinations using pollen from the ventilated storage containers, however, produced a large number of kernels, even when only a small portion of the surface area was ventilated for gas exchange. This very surprising result was the first time such high levels of seed set have been recorded using a seed-limiting dose of pollen preserved up to six days without cryo-preservation. In this experiment, there was no apparent benefit of ventilating more than 18% of the container surface area, corresponding to a surface area/volume ratio of about 0.14 cm⁻¹.

### Example 3

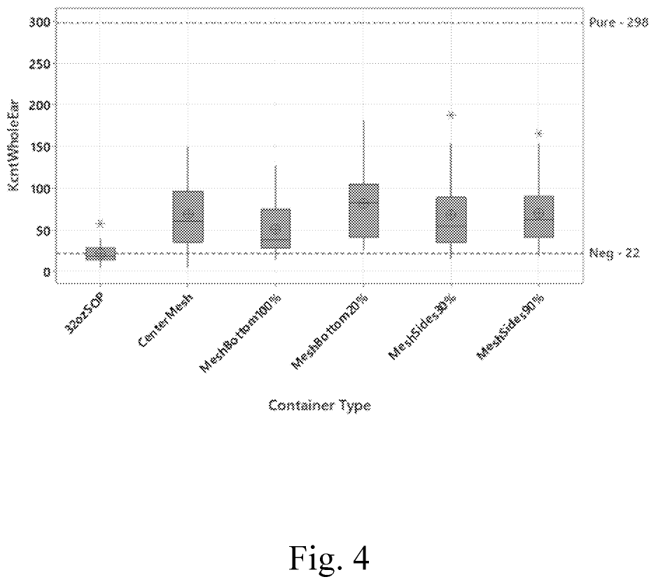

This experiment was conducted to confirm the results of Example 2 showing the benefit of passive ventilation during storage of the pollen-diluent mixture on seed set of field-grown plants. Pollen was collected from maize inbred WSS644, mixed with a diluent in a 4:1 ratio of diluent:fresh pollen (w/w), and maintained at 4 °C for 5 days in 32 oz (0.95 L) HDPE plastic containers prior to manual pollination ( 1/64th tsp applied by scoop and tube method) of receptive silks of maize inbred R5357. One set of containers (32 ozSOP) was unmodified and remained sealed without ventilation throughout storage. Other storage containers were ventilated to various degrees by replacing portions of the container walls with 50-micron nylon mesh to enable gas exchange between the pollen-diluent mix and the external environment. Ventilated modifications included: 100% Mesh Bottom Mesh, 20% Bottom Mesh, Center Column Mesh Column, 90% Mesh Sides, and 30% Mesh Sides. Physical parameters of the non-ventilated and partially ventilated storage vessels are listed in Table 1. FIG. 4 shows the results of this experiment. As shown in FIG. 4, pollen stored in the non-ventilated containers (Standard 32 oz), failed to produce any kernels after 5 days of storage at 4 °C. Pollinations using pollen from the ventilated storage containers, however, produced a large number of kernels, even when a small portion of the surface area was ventilated for gas exchange, confirming the results of Example 2.

### Example 4

This field experiment was conducted to determine whether the benefits of storage container ventilation could be secured in larger storage containers containing much greater volume/surface area ratios of the pollen-diluent mixture required for commercial applications. Pollen was collected from maize inbred WT944-6, mixed with a diluent in a 4:1 ratio of diluent:fresh pollen (w/w), and maintained at 4 °C for 2, 3, or 4 days prior to being applied to receptive silks of maize inbred R5357. Storage container volumes were 3.2 L, 13.2 L, and 18.9 L, corresponding to 120 oz, 3.5 gal, and 5.0 gal containers in the figures below. Container descriptions marked ‘GEX’ were ventilated with an internal porous column covered in 300 mesh cloth open to the atmosphere. The columns were 2.54 cm, 7.6 cm, and 10.2 cm in diameter for the 3.2 L, 13.2 L, and 18.9 L storage containers, respectively. The 18.9 L vessel contains sufficient pollen to pollinate approximately three acres of a typically hybrid maize seed production field (~96,000 female plants) using commercial application technology.

TABLE 2
Parameters of non-ventilated and partially ventilated storage vessels used to assess impact
of passive ventilation on viability of pollen stored in commercially relevant volumes.

| | | | | | | Vented |
|---|---|---|---|---|---|---|
| | | | | | Container | Surface Area: |
| | Container | Surface | Surface | | SA: Pollen- | Pollen- |
| | Surface | Area | Area | Pollen- | Diluent | Diluent |
| Storage | Area | Ventilated | Ventilated | Diluent | Volume | Volume |
| Container | (cm²) | (cm²) | (% total) | (g) | (cm⁻¹)* | (cm⁻¹)* |
| 120 oz (3.2 L) | 1380 | 0 | 0 | 1800 | 0.48 | 0 |
| 3.5 gal (13.2 L) | 2687 | 0 | 0 | 8580 | 0.19 | 0 |
| 120 oz (3.2 L) | 1380 | 203 | 15 | 1200 | 0.71 | 0.1 |
| GEX | | | | | | |
| 3.5 gal (13.2 L) | 2687 | 609 | 23 | 6912 | 0.24 | 0.05 |
| GEX | | | | | | |
| 5.0 gal (18.9 L) | 4060 | 853 | 21 | 9222 | 0.27 | 0.06 |
| GEX | | | | | | |

*assumes 620 g/L of pollen-diluent based on field measurements.

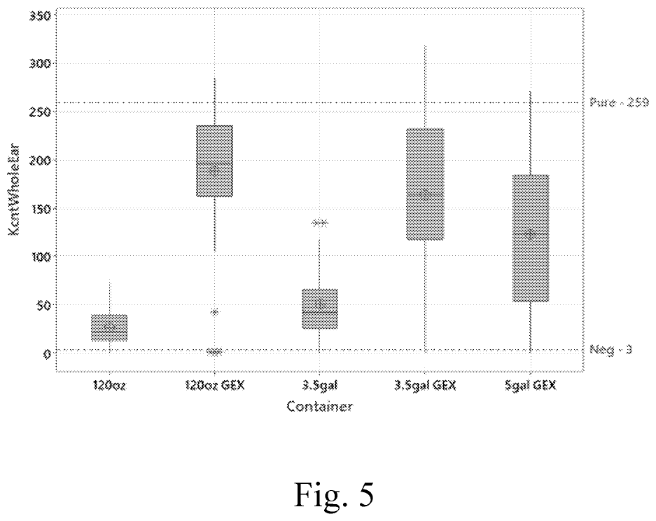

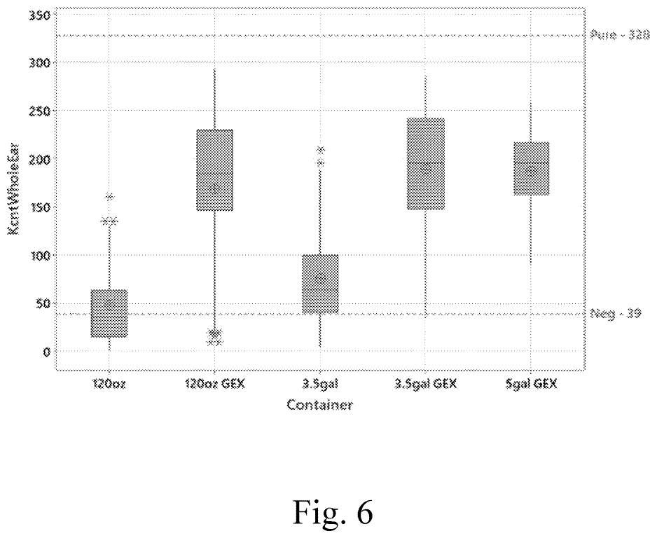

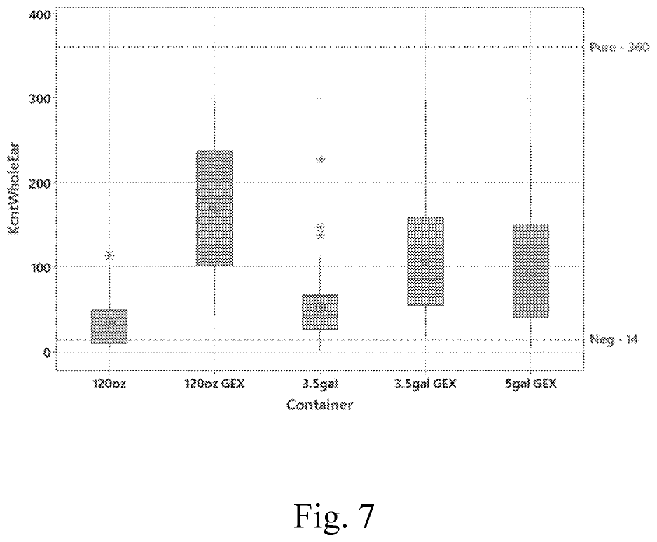

After two or three days of storage (D2, D3), plants were pollinated using a standard hand-pollination technique ( 1/64^{th} tsp applied by scoop and tube method); after the 4^{th} day of storage (D4), plants were pollinated by machine which delivers the pollen-diluent mix in an air stream to the plants. Both pollinating techniques deliver a limiting amount of pollen necessary to distinguish storage treatments on kernel set. Kernel numbers per ear were evaluated at least 14 days after pollination. FIG. 5 shows the kernel count per ear after two days of storage. FIG. 6 shows the kernel count per ear after three days of storage. FIG. 7 shows the kernel count after four days of storage. The results shown in FIGS. 5-7 show a dramatic positive impact of increasing the capacity for gas exchange (GEX) on kernel set across all days of storage, regardless of storage container volume. Since pollinated plants were of identical genetics and of similar silk age, these results clearly indicate container ventilation had direct positive impact of pollen viability in storage.

These surprising results indicated ventilation of the pollen-diluent mixture (GEX treatments) could dramatically improve seed set using pollen preserved in pollen/diluent volumes in excess of 9 kg. This is an entirely novel and unexpected result for the recalcitrant pollen produced by maize. Surprisingly, only a small fraction of the total container surface area of the storage container needed to provide ventilation to have a positive impact on the longevity of pollen viability required to affect successful pollination under field conditions.

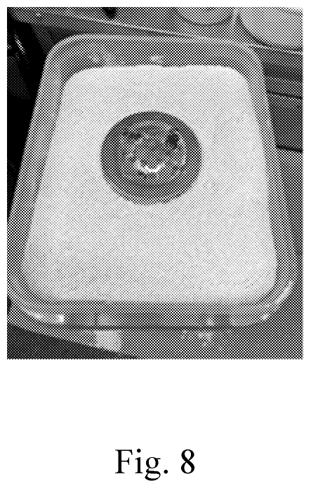

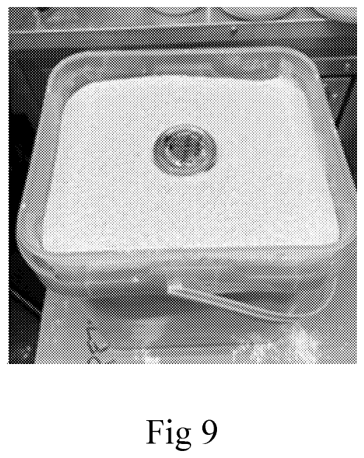

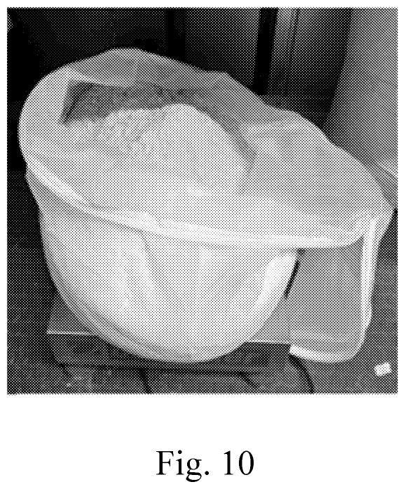

Shown in FIGS. 8-10 are examples of ventilated pollen storage systems used for commercially relevant volumes of pollen-diluent mixtures. FIG. 8 shows a solid sided plastic storage container containing 8 kg of pollen-diluent mixture fitted with a 2-L 10-micron central ventilation column, FIG. 9 shows a solid sided plastic storage container containing 9 kg of pollen-diluent mixture fitted with a 0.7-L 10-micron central ventilation column. FIG. 10 shows a collapsible and sealable 55-micron mesh bag containing 8 kg of pollen-diluent mixture.

TABLE 3
Parameters of non-ventilated and partially ventilated storage vessels used to assess impact
of passive ventilation on viability of pollen stored in commercially relevant volumes.

| | | | | | | | Vented Surface |
|---|---|---|---|---|---|---|---|
| | Container | | | Pollen- | | Vented | Area: |
| | Surface | Surface Area | | Diluent | Pollen per | Surface | Pollen-Diluent |
| Storage | Area | Ventilated | | Volume | Container | Area/Pollen | Volume |
| Container | (cm²) | (cm²) (% total) | | (cm³)* | (g) | (cm²/g) | (cm⁻¹)* |
| 15.7 L with | 4240 | 964 | 23 | 12903 | 1600 | 0.60 | 0.07 |
| 650 cm³ gas | | | | | | | |
| exchange | | | | | | | |
| column | | | | | | | |
| 15.7 L with | 4240 | 528 | 12 | 14516 | 1800 | 0.29 | 0.04 |
| 225 cm³ gas | | | | | | | |
| exchange | | | | | | | |
| column | | | | | | | |
| 55-micron | 3633 | 1163 ** | 32 | 12903 | 1600 | 0.73 | 0.13 |
| mesh bags | | | | | | | |

*620 g/L of pollen-diluent based on field measurements.
** 55-micron mesh with approximately 32% open surface.

### Example 5

The following set of field experiments were conducted to determine whether flexible ventilated containers containing commercially relevant volumes of pollen-diluent mixture improved pollen viability in storage. The trials involved four male inbreds as pollen donors (WT944, ILLHO, WSS644, and PGH47) and four male-sterile female inbreds as pollen recipients (R5357, T2721, W8039, and F0457) tested in various combinations across six independent experiments. Fresh pollen was collected mechanically from each male inbred and mixed with a diluent in a 4:1 ratio of diluent:pollen (w/w) and stored in either a solid sided 15.7-L plastic storage container fitted with a 2-L 10-micron ventilation column or in a collapsible and sealable 55-micron mesh bag shown in FIG. 8. The pollen-diluent mixtures were maintained at 3.5±0.6 °C and 79±5.7% RH for up to 3 days. After 1, 2, or 3 days of storage, pollen-diluent mixture was applied manually in amounts that limit kernel set ( 1/64^{th} tsp applied by scoop and tube method) to receptive silks of a male-sterile recipient inbred approximately three days after silks first emerged (3 replicates of 10 plants per treatment, N=30). Pollen viability was assessed in terms of seed set per plant measured at least 14 days after pollination.

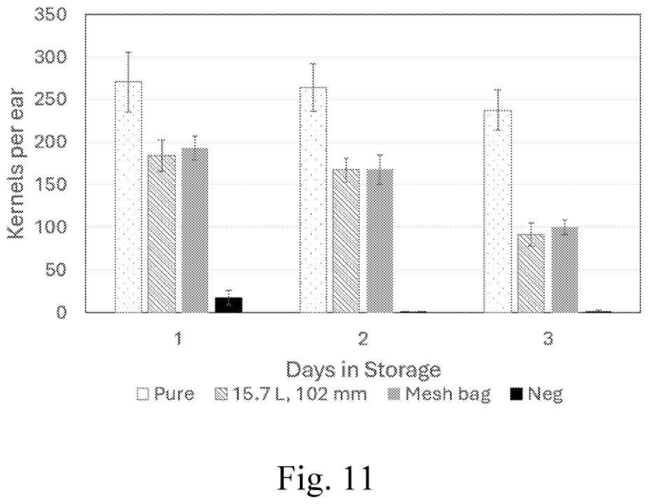

FIG. 11 shows the combined results for the six field experiments comparing seed set on maize ears pollinated with the pollen-diluent mixture stored in 15.7-L ventilated rigid containers or in flexible 55-micron mesh bags. Results for plants pollinated with pure, freshly collected pollen (Pure) and unpollinated plants (Neg) are included for comparison. Each bar represents the average±standard error of the six field experiments having three replications of 10 plants each (N=180 plants). Both the solid-sided centrally ventilated container and the flexible mesh bag were equally effective in maintaining pollen viability in storage. All combinations of male pollen source and female recipient inbred produced remarkably similar results for the two storage systems. As expected, the more kernels were produced in the Pure treatment, which received a saturating dose of pollen four times greater than that applied with pollen-diluent mixtures. Kernels per ear resulting from pollinations using pollen stored in the solid sided container after 1, 2, and 3 days of storage were approximately 68%, 63%, and 39%, respectively, of the Pure treatment. Likewise, kernels per ear resulting from pollinations using pollen stored in the flexible mesh bag after 1, 2, and 3 days of storage were approximately 71%, 64%, and 42%, respectively, of the Pure treatment. These results confirm that recalcitrant maize pollen can be stored successfully in both rigid and flexible mesh storage containers providing 0.07 to 0.13 cm² of ventilated surface area per cm³ of pollen-diluent mixture.

### Example 6

This study was conducted on field-grown plants to evaluate the viability of maize pollen stored in commercial volumes required for field-scale application. Approximately 10 kg of fresh pollen was collected mechanically from ALEXHO hybrids and mixed mechanically with 40 kg of a diluent (4:1 diluent:pollen ratio by weight). The pollen-diluent mixture was divided among six storage containers; one containing 1.5 kg in a non-ventilated rigid container, and others containing 2, 4, 8, 10, or 12 kg in the flexible 55-micron mesh bags described in FIG. 10 and Table 5. All storage vessels were maintained in a controlled environment at 3.9±1.7 °C and 74±4.3% relative humidity (RH). Samples were taken after two and four days of storage for metered (pollen limiting) hand pollinations on male-sterile female plants of inbred R5357, with 10 plants x3 replications per treatment (N=30). Each plant was pollinated manually ( 1/64^{th} tsp applied by scoop and tube method) approximately three days silks first emerged. Pollen viability was assessed in terms of seed set on plants measured at least 14 days after pollination. 1/64 tsp of pollen-diluent mixture at 4-1 ratio contains approximately 2500 grains of maize pollen.

TABLE 5 a
Parameters of rigid and flexible container configurations used to assess impact of controlled
gas exchange on longevity of commercially relevant volumes of maize pollen in storage.

| | | | | | | | | Vented |
|---|---|---|---|---|---|---|---|---|
| | | | | | | | | Surface |
| | | | | | | Pollen - | | Area/ |
| | Container | | | Pollen- | Pollen | Diluent | Vented | Pollen- |
| | Surface | Vented | | Diluent | per | per | Surface | Diluent |
| Storage | Area | Surface Area | | Volume | Container | container | Area/Pollen | Volume |
| Container | (cm²) | (cm²) (% total) | | (cm³)* | (g) | (g) | (cm²/g) | (cm⁻¹)* |
| 3.5-L | 1380 | 0 | 0 | 2419 | 300 | 1500 | 0.0 | 0.0 |
| sealed | | | | | | | | |
| container | | | | | | | | |
| 55-micron | 2619 | 838 | 32** | 3225 | 400 | 2000 | 2.09 | 0.26 |
| mesh bag | | | | | | | | |
| 55-micron | 2958 | 946 | 32** | 6452 | 800 | 4000 | 1.18 | 0.15 |
| mesh bag | | | | | | | | |
| 55-micron | 3635 | 1163 | 32** | 12903 | 1600 | 8000 | 0.73 | 0.13 |
| mesh bag | | | | | | | | |
| 55-micron | 3974 | 1272 | 32** | 16129 | 2000 | 10000 | 0.64 | 0.08 |
| mesh bag | | | | | | | | |
| 55-micron | 4312 | 1380 | 32** | 19355 | 2400 | 12000 | 0.58 | 0.07 |
| mesh bag | | | | | | | | |

*620 g/L of pollen-diluent mixture based on field measurements.
**55-micron mesh with approximately 32% open surface.

TABLE 5 b
Parameters of rigid and flexible storage vessel used to assess impact of controlled gas
exchange on longevity of commercially relevant volumes of maize pollen in storage.

| | Day 2 | Day 2 | Day 4 | Day 4 | Day 2 | Day 2 | Day 4 | Day 4 |
|---|---|---|---|---|---|---|---|---|
| | Mean | Median | Mean | Median | pollination | pollination | pollination | pollination |
| Storage | Kernel | Kernel | Kernel | Kernel | efficiency | efficiency | efficiency | efficiency |
| Container | Count | Count | Count | Count | (mean) | (Median) | (mean) | (Median) |
| 3.5-L | 62.30 | 61.50 | 65.60 | 64.00 | 40.1284109 | 40.65041 | 38.10976 | 39.0625 |
| sealed | | | | | | | | |
| container | | | | | | | | |
| 55- | 176.20 | 183.00 | 152.90 | 154.00 | 14.1884222 | 13.6612 | 16.35056 | 16.23377 |
| micron | | | | | | | | |
| mesh bag | | | | | | | | |
| (2 kg) | | | | | | | | |
| 55- | 147.20 | 165.00 | 142.60 | 144.00 | 16.9836957 | 15.15152 | 17.53156 | 17.36111 |
| micron | | | | | | | | |
| mesh bag | | | | | | | | |
| (4 kg) | | | | | | | | |
| 55- | 146.70 | 166.00 | 124.00 | 134.50 | 17.0415815 | 15.06024 | 20.16129 | 18.58736 |
| micron | | | | | | | | |
| mesh bag | | | | | | | | |
| (8 kg) | | | | | | | | |
| 55- | 120.50 | 121.50 | 139.30 | 142.50 | 20.746888 | 20.57613 | 17.94688 | 17.54386 |
| micron | | | | | | | | |
| mesh bag | | | | | | | | |
| (10 kg) | | | | | | | | |
| 55- | 126.20 | 131.50 | 110.30 | 96.00 | 19.8098257 | 19.01141 | 22.66546 | 26.04167 |
| micron | | | | | | | | |
| mesh bag | | | | | | | | |
| (12 kg) | | | | | | | | |
| 55- | 126.20 | 131.50 | 110.30 | 96.00 | 40.1284109 | 40.65041 | 38.10976 | 39.0625 |
| micron | | | | | | | | |
| mesh bag | | | | | | | | |
| (12 kg) | | | | | | | | |

*620 g/L of pollen-diluent mixture based on field measurements.
**55-micron mesh with approximately 32% open surface

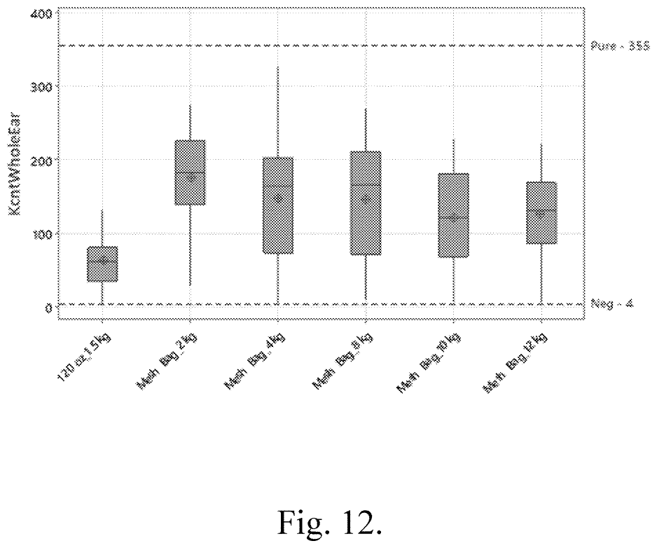

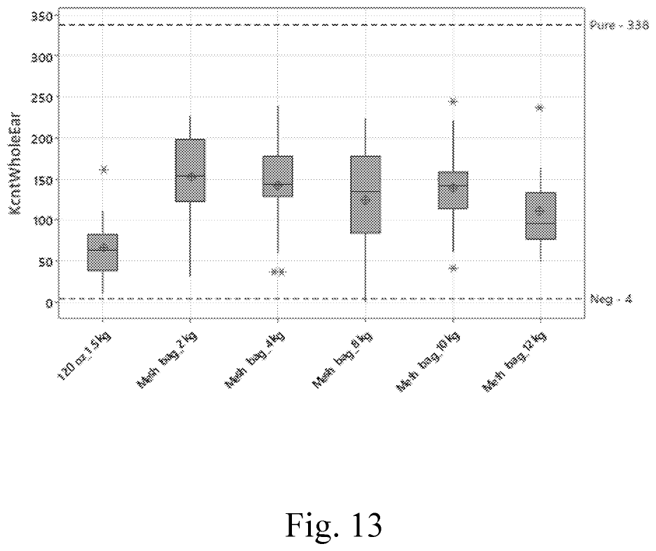

FIGS. 12 and 13 present box plots of kernels per ear resulting from pollinations using ALEXHO hybrid pollen stored for two days (FIG. 12) and four days (FIG. 13) in various containers. The symbol within each box represents the mean of three replications of 10 plants each (n=30). As observed in Example 5, pollen viability assessed in terms of kernel set was much greater in the flexible mesh bags than in the non-ventilated container after two days of storage. The capacity of the flexible mesh bags to maintain the viability of pollen in quantities of up to 12 kg (19.4 L) of pollen-diluent mixture for four days, however, is unprecedented. This surprising result presents a new market opportunity for the management of recalcitrant pollen in commercial seed, grain, and fruit production dependent upon timely delivery of pollen to receptive female plants.

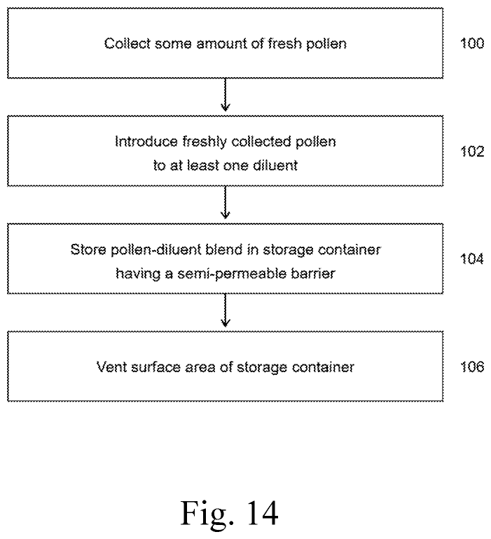

FIG. 14 is a flow chart view of an embodiment of the methodology of the present invention. In FIG. 14, an embodiment of the methodology of the present invention begins by collecting some amount of fresh pollen at step 100. Next the freshly collected pollen is introduced to at least one diluent at step 102. The pollen-diluent blend is then stored in a storage container having a semi-permeable barrier at step 104. Finally, the surface area of the storage container is vented during storage at step 106.

What is claimed is:

1. A method of preserving the integrity and viability of live pollen comprising:
a) Introducing freshly collected pollen to at least one diluent to create a pollen-diluent mixture;
b) Storing said pollen-diluent mixture in a storage container, wherein said storage container comprises a semi-permeable barrier over at least a portion of the surface area of said storage container; and
c) Venting some portion of the surface area of said storage container during storage, wherein said vented portion of said surface area comprises at least 10% of the total surface area of said storage container, wherein said pollen is capable of pollinating plants after a storage period of at least two days, wherein said pollen has a level of viability such that less than 500 pollen grains are required per each successful fertilization event.

2. The method of claim 1 further comprising storing said storage container containing said pollen-diluent mixture in a controlled environment for a period of time.

3. The method of claim 1 wherein the weight of said pollen is at least 300 grams.

4. The method of claim 3 wherein the weight of said pollen is between 300 grams and 2400 grams.

5. The method of claim 1 wherein the weight of said pollen-diluent mixture is at least 1500 grams.

6. The method of claim 5 wherein the weight of said pollen-diluent mixture is between 1500 grams and 12,000 grams.

7. The method of claim 1 wherein said storage container comprises a surface area to pollen weight ratio of at least 0.5 square centimeters per gram.

8. The method of claim 7 wherein said storage container comprises a surface area to pollen weight ratio between 0.5 to 7 square centimeters per gram.

9. The method of claim 1 wherein said storage container comprises a vented surface area to pollen-diluent mixture volume ratio of at least 0.03 cm⁻¹.

10. The method of claim 9 wherein said storage container comprises a vented surface area to pollen volume ratio between 0.03 cm⁻¹ to 0.44 cm⁻¹.

11. The method of claim 1 wherein said storage container further comprises a variable surface area, wherein said variable surface area depends on the volume of pollen-diluent mixture stored in said storage container.

12. The method of claim 1 wherein said controlled environment comprises a relative humidity between 60-100%.

13. The method of claim 12 wherein said controlled environment is configured to maintain a pollen moisture content of between 40-60%.

14. A method of preserving the integrity and viability of live pollen comprising:
a) collecting some amount of fresh pollen;
b) Introducing said fresh pollen to at least one diluent to create a pollen-diluent mixture;
c) Storing said pollen-diluent mixture in a mesh bag, wherein said mesh bag comprises a semi-permeable barrier over at least a portion of the surface area of said mesh bag, and
d) Venting some portion of the surface area of said mesh bag during storage.

15. The method of claim 14 wherein said mesh bag further comprises a variable surface area, wherein said variable surface area depends on the volume of pollen-diluent mixture stored in said mesh bag.

16. The method of claim 14 wherein the mesh bag comprises a surface area to pollen weight ratio of at least 1.5 square centimeters per gram.

17. The method of claim 16 wherein the mesh bag comprises a surface area to pollen weight ratio of between 1.5 to 7 square centimeters per gram.

18. A method of preserving the integrity and viability of live pollen comprising:
a) Introducing freshly collected pollen to at least one diluent to create a pollen-diluent mixture;
b) Storing said pollen-diluent mixture in a mesh bag, wherein said mesh bag comprises a semi-permeable barrier over at least a portion of the surface area of said mesh bag;
c) Venting some portion of the surface area of said mesh bag during storage; and
d) Applying said pollen-diluent mixture to a plant such that said pollen successfully pollinates said plant.

19. The method of claim 18 wherein the application step results in a pollination efficiency of less than 500 grains per seed.

20. The method of claim 19 wherein the application step results in a pollination efficiency of less than 200 grains per seed.
